(19) United States
(12) Patent Application Publication
(10) Pub. No.: US 2024/0093205 A1
(43) Pub. Date: Mar. 21, 2024

(54) DUAL CHECKPOINT INHIBITOR APTAMER BASED THERAPEUTICS

(71) Applicant: Regen Biopharma, Inc., La Mesa, CA (US)

(72) Inventors: Thomas ICHIM, San Diego, CA (US); David KOOS, La Mesa, CA (US); Harry LANDER, La Mesa, CA (US)

(73) Assignee: Regen Biopharma, Inc., La Mesa, CA (US)

(21) Appl. No.: 18/470,300

(22) Filed: Sep. 19, 2023

Related U.S. Application Data
(60) Provisional application No. 63/406,160, filed on Sep. 13, 2022.

(51) Int. Cl. C12N 15/115

(52) U.S. Cl. CPC C12N 15/115; C12N 2310/16; C12N 2310/531

(57) ABSTRACT

Disclosed are aptamers designed to bind immunological checkpoint molecules while concurrently suppressing tumor associated gene expression through delivery of DNA molecules encoding short hairpin DNA. In one embodiment the invention provides an aptamer capable of binding PD-1 and/or PD-1 ligand while concurrently possessing ability to induce RNA interference to one more multiple other immune inhibitory and/or oncogenesis related genes. In one embodiment such a dual-targeting aptamer is utilized to induce systemic tumor abscopal effect.

### CROSS-REFERENCE TO RELATED APPLICATIONS

This application claims priority back to U.S. Provisional Application No. 63/406,160, titled “Dual Checkpoint Inhibitor Aptamer Based Therapeutics”, filed Sep. 13, 2022, which is hereby incorporated by reference in its entirety.

### FIELD OF THE INVENTION

The teachings herein relate to new therapeutic aptamers having dual activity in that they are capable of binding to a molecule associated with inhibition of immunological activity, while concurrently possessing a nucleic acid sequence capable of generating a molecule or series of molecules which induce RNA interference towards cancer promoting and/or immune suppressive molecules.

### BACKGROUND

Despite significant progress in the area of chemotherapeutic development, substantial improvements have not been made in survival of solid tumors. One promising therapeutic in development is the utilization of the immune system to seek and destroy cancer. Unfortunately, numerous tumor derived molecules actively suppress immunity to tumors.

It is known in the art that aptamers are short, single-stranded nucleic acid oligomers or peptides that can bind to target molecules in a specific manner. Aptamers are typically selected from a large random pool of candidates in an iterative process. More recently, aptamers have been successfully selected in cells, in-vivo and in-vitro, this allows for creation of numerous potential molecules for therapeutic processes. The selection of aptamers, their structure-function relationship, and their mechanisms of action are all poorly-understood. Although more than 100 aptamer structures have been solved and reported, almost no recurring structural motifs have been identified.

A variety of different aptamer selection processes have been described for enriching aptamer libraries for aptamers capable of binding to a particular target. Certain of the binding aptamers identified from such binding-enriched pools have later been determined to be capable of mediating a functional effect on a cell. However, the fact that an aptamer binds to a cell does not mean that it will induce a desirable cellular function. For example, many aptamers that merely bind to a particular target cell will have no effect on that cell's function, or may induce a cell function completely different from the one that is desired. Moreover, functional aptamers that bind weakly to a target cell and/or that bind to antigens that are expressed at low levels on the surface of the target cell will not be enriched by conventional aptamer selection processes.

### SEQUENCE LISTING

(SEQ ID NO: 1)
CGGCAAGCAUUACGGUGUC
(SEQ ID NO: 2)
GCAUCGACAACGUGUGCGA
(SEQ ID NO: 3)
cugguuccccagcugucag
(SEQ ID NO: 4)
ggaagagacuuuguccaca
(SEQ ID NO: 5)
ggaagagactttgtccacat

### SUMMARY

Preferred embodiments are directed to compositions comprising an aptamer or aptamer-like composition capable of binding to a molecule associated with inhibition of immunological activity in a manner as to inhibit activity of said molecule associated with inhibition of immunological activity, while concurrently possessing a nucleic acid sequence capable of generating a molecule or series of molecules which induce RNA interference towards cancer promoting and/or immune suppressive molecules.

Preferred embodiments include compositions wherein said molecule is capable of inhibition of the Adenosine A2A receptor.

Preferred embodiments include compositions wherein said molecule is capable of inhibition of the Adenosine A2B receptor.

Preferred embodiments include compositions wherein said molecule is capable of inhibition of CD276.

Preferred embodiments include compositions wherein said molecule is capable of inhibition of VTCN1.

Preferred embodiments include compositions wherein said molecule is capable of inhibition of BTLA.

Preferred embodiments include compositions wherein said molecule is capable of inhibition of CTLA-4.

Preferred embodiments include compositions wherein said molecule is capable of inhibition of Indolamine 2,3 deoxygenase.

Preferred embodiments include compositions wherein said molecule is capable of inhibition of Killer-cell Immunoglobulin-like Receptor.

Preferred embodiments include compositions wherein said molecule is capable of inhibition of LAG-3.

Preferred embodiments include compositions wherein said molecule is capable of inhibition of NOX-2.

Preferred embodiments include compositions wherein said molecule is capable of inhibition of PD-1.

Preferred embodiments include compositions wherein said molecule is capable of inhibition of TIM-3.

Preferred embodiments include compositions wherein said molecule is capable of inhibition of VISTA.

Preferred embodiments include compositions wherein said molecule is capable of inhibition of ILT3.

Preferred embodiments include compositions wherein said molecule is capable of inhibition of ILT4.

Preferred embodiments include compositions wherein said molecule is capable of inhibition of TGF-beta receptor.

Preferred embodiments include compositions wherein said molecule is capable of inhibition of interleukin-10 receptor.

Preferred embodiments include compositions wherein said molecule is capable of inhibition of interleukin-4 receptor.

Preferred embodiments include compositions wherein said molecule is capable of inhibition of interleukin-13 receptor.

Preferred embodiments include compositions wherein said molecule is capable of inhibition of interleukin-17 receptor.

Preferred embodiments include compositions wherein said molecule is capable of inhibition of interleukin-20 receptor.

Preferred embodiments include compositions wherein said molecule is capable of inhibition of VEGF receptor.

Preferred embodiments include compositions wherein said molecule is capable of inhibition of interleukin-24.

Preferred embodiments include compositions wherein said molecule is capable of inhibition of interleukin-27.

Preferred embodiments include compositions wherein said molecule is capable of inhibition of interleukin-35.

Preferred embodiments include compositions wherein said molecule is capable of inhibition of Siglec-3.

Preferred embodiments include compositions wherein said molecule is capable of inhibition of Siglec-5.

Preferred embodiments include compositions wherein said molecule is capable of inhibition of Siglec-7.

Preferred embodiments include compositions wherein said molecule is capable of inhibition of galectin-1.

Preferred embodiments include compositions wherein said molecule is capable of inhibition of galectin-3.

Preferred embodiments include compositions wherein said molecule is capable of inhibition of galectin-9.

Preferred embodiments include compositions wherein said molecule is capable of inhibition of PD-L1.

Preferred embodiments include compositions wherein said molecule is capable of inhibition of FCγRIIb.

Preferred embodiments include compositions wherein said molecule is capable of inhibition of GP49.

Preferred embodiments include compositions wherein said molecule contains DNA molecules which are capable of transcribing short hairpin RNA.

Preferred embodiments include compositions wherein said short hairpin RNA contains a stem loop.

Preferred embodiments include compositions wherein said short hairpin RNA induces RNA interference.

Preferred embodiments include compositions wherein said RNA interference is induced to inhibit expression of survivin.

Preferred embodiments include compositions wherein said RNA interference is induced to inhibit expression of livin.

Preferred embodiments include compositions wherein said RNA interference is induced to inhibit expression of CD161.

Preferred embodiments include compositions wherein said RNA interference is induced to inhibit expression of CD137.

Preferred embodiments include compositions wherein said RNA interference is induced to inhibit expression of an oncogene.

Preferred embodiments include compositions wherein said oncogene is selected from a group comprising of: a) abl, b) Af4/hrx, c) akt-2, d) alk, e) alk/npm, f) aml1, g) aml1/mtg8, h) bcl-2, 3, 6, i) bcr/abl, j) c-myc, k) dbl, l) dek/can, m) E2A/pbx1, n) egfr, o) enl/hrx, p) erg/TLS, q) erbB, r) erbB-2, s) ets-1, t) ews/fli-1, u) fms, v) fos, w) fps, x) gli, y) gsp, z) gsp, aa) HER2/new, ab) hoxl1, ac) hst, ad) IL-3, ae) int-2, af) jun, ag) kit, ah) KS3, ai) K-sam, aj) Lbc, ak) lck, al) Imo1, Imo-2, am) L-myc, an) lyl-1, ao) lyt-10, ap) lyt-10/C alpha 1, aq) mas, ar) mdm-2, as) mll, at) mos, au) mtg8/aml1, av) myb, aw) MYH11, ax) new, ay) N-myc, az) ost, ba) pax-5, bb) pbx1/E2a, bc) pim-1, bd) PRAD-1, be) raf, bf) RAR/PML, bg) Ras H, K, N, bh) rel/nrg, bi) ret, bj) rhom1, rhom2, bk) ros, bl) ski, bm) sis, bn) set/can, bo) src, bp) Tal1, tal2, bq) tan-1, br) Tiam1, bs) TSC2, and bt) trk.

Preferred embodiments include compositions wherein CpG motifs are enriched in said aptamer so as to increase immunogenicity of said aptamer.

Preferred embodiments include compositions wherein said CpG motifs activate TLR9.

Preferred embodiments include compositions wherein DNA encodes an RNA molecule in whole or in part expressing the sequence at least 10% homology to the sequence: CGGCAAGCAUUACGGUGUC (SEQ ID NO: 1) capable of inhibiting NR2F6.

Preferred embodiments include compositions wherein DNA encodes an RNA molecule in whole or in part expressing the sequence at least 10% homology to the sequence: GCAUCGACAACGUGUGCGA (SEQ ID NO: 2) capable of inhibiting NR2F6.

Preferred embodiments include compositions wherein DNA encodes an RNA molecule in whole or in part expressing the sequence at least 10% homology to the sequence: CUGGUUCCCCAGCUGUCAG (SEQ ID NO: 3) capable of inhibiting livin.

Preferred embodiments include compositions wherein DNA encodes an RNA molecule in whole or in part expressing the sequence at least 10% homology to the sequence: GGAAGAGACUUUGUCCACA (SEQ ID NO: 4) capable of inhibiting livin.

Preferred embodiments include compositions wherein DNA encodes an RNA molecule in whole or in part expressing the sequence at least 10% homology to the sequence: GGAAGAGACTTTGTCCACAT (SEQ ID NO: 5) capable of inhibiting survivin.

Preferred embodiments include compositions wherein said molecules are used in conjunction with an NF-kappa B inhibitor.

### DETAILED DESCRIPTION OF THE INVENTION

The invention seeks to address the need in the art for novel cancer therapeutics. In one embodiment the invention provides an aptamer enriched from aptamer libraries for aptamers that mediate a desired cellular function of immune stimulation and/or cancer inhibition. Importantly, such methods and compositions would enable the direct identification of aptamers able to modulate a desirable functional effect on a target cell of interest, which would have a profound impact on aptamer therapeutics. In some embodiments aptamers are generated to block activation of immunological checkpoints by direct binding, while concurrently inserting into said aptamers sequences capable of inducing RNA interference to intracellular targets such as oncogenes.

In the present invention, “cancer” is a generic term for diseases caused by cells having an aggressive characteristic in which cells divide and grow ignoring normal growth limits, an invasive characteristic in which cells penetrate surrounding tissues, and a metastatic characteristic in which cells spread to other parts of the body. The cancer is preferably at least one selected from the group consisting of gastric cancer, breast cancer, lung cancer, liver cancer, blood cancer, bone cancer, pancreatic cancer, skin cancer, head or neck cancer, cutaneous or intraocular melanoma, uterine sarcoma, ovarian cancer, rectal cancer, anal cancer, colon cancer, fallopian tube carcinoma, endometrial carcinoma, cervical cancer, small intestine cancer, endocrine cancer, thyroid cancer, parathyroid cancer, adrenal cancer, soft tissue tumor, urethral cancer, prostate cancer, bronchogenic cancer and bone marrow tumor, but is not limited thereto.

In the present invention, “cancer stem cells” refers to cells having the ability to generate a tumor. Cancer stem cells have the same characteristics as normal stem cells, and specifically have the ability to give rise to all cell types found in a specific cancer sample. That is, cancer stem cells are tumorigenic differently from cancer cells that do not form a tumor. Cancer stem cells generate a tumor in various cell types through the self-renewal and differentiation capacity, which are characteristics of stem cells. In addition, cancer stem cells are distinct from other populations in the tumor and cause recurrence and metastasis by generating a new tumor. Thus, the development of a specific treatment method targeting cancer stem cells may increase the survival rate of cancer patients.

In the present invention, “nucleic acid” refers to deoxyribonucleotides or ribonucleotides and polymers thereof in either single-, double- or multiple-stranded form, or complements thereof. The term “polynucleotide” refers to a linear sequence of nucleotides. The term “nucleotide” typically refers to a single unit of a polynucleotide, i.e., a monomer. Nucleotides can be ribonucleotides, deoxyribonucleotides, or modified versions thereof. Examples of polynucleotides contemplated herein include single and double stranded DNA, single and double stranded RNA (including siRNA), and hybrid molecules having mixtures of single and double stranded DNA and RNA. Nucleic acids can be linear or branched. For example, nucleic acids can be a linear chain of nucleotides or the nucleic acids can be branched, e.g., such that the nucleic acids comprise one or more arms or branches of nucleotides. Optionally, the branched nucleic acids are repetitively branched to form higher ordered structures such as dendrimers and the like. The terms also encompass nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphodiester derivatives including, e.g., phosphoramidate, phosphorodiamidate, phosphorothioate (also known as phosphothioate), phosphorodithioate, phosphonocarboxylic acids, phosphonocarboxylates, phosphonoacetic acid, phosphonoformicacid, methyl phosphonate, boron phosphonate, or O-methylphosphoroamidite linkages (see Eckstein, Oligonucleotides and Analogues: A Practical Approach, Oxford University Press), and peptide nucleic acid backbones and linkages. Other analog nucleic acids include those with positive backbones, non-ionic backbones, modified sugars, and non-ribose backbones (e.g. phosphorodiamidate morpholino oligos or locked nucleic acids (LNA)), including those described in U.S. Pat. Nos. 5,235,033 and 5,034,506, and Chapters 6 and 7, ASC Symposium Series 580, Carbohydrate Modifications in Antisense Research, Sanghui & Cook, eds. Nucleic acids containing one or more carbocyclic sugars are also included within one definition of nucleic acids. Modifications of the ribose-phosphate backbone may be done for a variety of reasons, e.g., to increase the stability and half-life of such molecules in physiological environments or as probes on a biochip. Mixtures of naturally occurring nucleic acids and analogs can be made, alternatively, mixtures of different nucleic acid analogs, and mixtures of naturally occurring nucleic acids and analogs may be made. In embodiments, the internucleotide linkages in DNA are phosphodiester, phosphodiester derivatives, or a combination of both.

The aptamer that specifically binds to cancer cells and/or cancer stem cells of the present invention has effects of reducing cell adhesion ability, cell proliferation, drug resistance and cell migration, which are characteristics of cancer stem cells, and may be thus used in various ways in the fields of cancer diagnosis, prognosis prediction, and treatment. In some embodiments the aptamers are capable of blocking activation of immunological checkpoints while delivering molecules that induce RNA interference to cancer cells.

The term “aptamer” as provided herein refers to oligonucleotides (e.g., short oligonucleotides or deoxyribonucleotides), that bind (e.g., with high affinity and specificity) to proteins, peptides, and small molecules. An aptamer may be referred to as an oligonucleotide-based target binding moiety. Aptamers may be RNA or DNA. Aptamers may have secondary or tertiary structure and, thus, may be able to fold into diverse and intricate molecular structures.

For the practice of the invention, aptamers can be selected in vitro from very large libraries of randomized sequences by the process of systemic evolution of ligands by exponential enrichment (SELEX as described in Ellington A D, Szostak J W (1990) In vitro selection of RNA molecules that bind specific ligands. Nature 346:818-822, Tuerk C, Gold L (1990) Systematic evolution of ligands by exponential enrichment: RNA ligands to bacteriophage T4 DNA polymerase. Science 249:505-510) or by developing SOMAmers (slow off-rate modified aptamers) (Gold L et al. (2010) Aptamer-based multiplexed proteomic technology for biomarker discovery. PLoS ONE 5(12):e15004). Applying the SELEX and the SOMAmer technology includes for instance adding functional groups that mimic amino acid side chains to expand the aptamer's chemical diversity. As a result, high affinity aptamers for a protein may be enriched and identified. Aptamers may exhibit many desirable properties for targeted drug delivery, such as ease of selection and synthesis, high binding affinity and specificity, low immunogenicity, and versatile synthetic accessibility. Anticancer agents (e.g., chemotherapy drugs, toxins, and siRNAs) may be successfully delivered to cancer cells in vitro using aptamers. Aptamers are nucleic acid molecules characterised by the ability to bind to a target molecule with high specificity and high affinity. Almost every aptamer identified to date is a non-naturally occurring molecule. Aptamers may be DNA or RNA molecules and may be single stranded or double stranded. The aptamer may comprise chemically modified nucleotides or nucleosides, for example in which the sugar and/or phosphate and/or base is chemically modified. Such modifications may improve the stability of the aptamer or make the aptamer more resistant to degradation. The aptamers of the present invention may include chemical modifications as described herein such as a chemical substitution at a sugar position, a phosphate position, and/or a base position of the nucleic acid including, for example, incorporation of a modified nucleotide, incorporation of a capping moiety (e.g. 3′ capping), conjugation to a high molecular weight, non-immunogenic compound (e.g. polyethylene glycol (PEG)), conjugation to a lipophilic compound, substitutions in the phosphate backbone. Base modifications may include 5-position pyrimidine modifications, modifications at exocyclic amines, substitution of 4-thiouridine, substitution of 5-bromo- or 5-iodo-uracil, backbone modifications. Sugar modifications may include 2′-amine nucleotides (2′-NH.sub.2), 2′-fluoro nucleotides (2′-F), and 2′-O-methyl (2′-OMe) nucleotides. A wide range of nucleotide, nucleoside, base and phosphate modifications are known to those or ordinary skill in the art, e.g., as described in Eaton et al., Bioorganic & Medicinal Chemistry, Vol. 5, No. 6, pp 1087-1096, 1997. Aptamers may be synthesized by methods which are well known to the skilled person. For example, aptamers may be chemically synthesized, e.g., on a solid support. Solid phase synthesis may use phosphoramidite chemistry. Briefly, a solid supported nucleotide is detritylated, then coupled with a suitably activated nucleoside phosphoramidite to form a phosphite triester linkage. Capping may then occur, followed by oxidation of the phosphite triester with an oxidant, typically iodine. The cycle may then be repeated to assemble the aptamer (e.g., see Sinha, N. D., Biernat, J., McManus, J., Koster, H. Nucleic Acids Res. 1984, 12, 4539, and Beaucage, S. L., Lyer, R. P. (1992). Tetrahedron 48 (12): 2223). Aptamers can be thought of as the nucleic acid equivalent of monoclonal antibodies and often have K.sub.d's in the nM or pM range, e.g. less than one of 500 nM, 100 nM, 50 nM, 10 nM, 1 nM, 500 pM, 100 pM. As with monoclonal antibodies, they may be useful in virtually any situation in which target binding is required, including use in therapeutic and diagnostic applications, in vitro or in vivo. In vitro diagnostic applications may include use in detecting the presence or absence of a target molecule.

In one embodiment the invention provides methods for generating a functionally enriched population of aptamers capable of binding immunological checkpoints on cells while concurrently inducing RNA interference to a secondary target. Immunological checkpoints include

In certain embodiments, the method comprises: (a) contacting target cells with a plurality of particles on which are immobilized a library of aptamer clusters (“aptamer cluster particles”), wherein at least a subset of the immobilized aptamer clusters bind to at least a subset of the target cells to form cell-aptamer cluster particle complexes, (b) incubating the cell-aptamer cluster particle complexes for a period of time sufficient for at least some of the target cells in the cell-aptamer cluster particle complexes to undergo a cell function, (c) detecting the cell-aptamer cluster particle complexes undergoing the cell function, (d) separating cell-aptamer cluster particle complexes comprising target cells undergoing the cell function detected in step (c) from other cell-aptamer cluster particle complexes, and (e) amplifying the aptamers in the separated cell-aptamer cluster particle complexes to generate a functionally enriched population of aptamers. In some embodiments, steps (c) and (d) are performed using a flow cytometer.

In one embodiment of the invention, administration of aptamers disclosed herein is performed together with other adjuvant therapies. One form of adjuvant therapies includes administration with compounds having a cytostatic or anti-neoplastic effect (“cytostatic compound”). Cytostatic compounds included in the present invention comprise, but are not restricted to (i) antimetabolites, such as cytarabine, fludarabine, 5-fluoro-2′-deoxyuridine, gemcitabine, hydroxyurea or methotrexate, (ii) DNA-fragmenting agents, such as bleomycin, (iii) DNA-crosslinking agents, such as chlorambucil, cisplatin, cyclophosphamide or nitrogen mustard, (iv) intercalating agents such as adriamycin (doxorubicin) or mitoxantrone, (v) protein synthesis inhibitors, such as L-asparaginase, cycloheximide, puromycin or diphteria toxin, (vi) topoisomerase I poisons, such as camptothecin or topotecan, (vii) topoisomerase II poisons, such as etoposide (VP-16) or teniposide, (viii) microtubule-directed agents, such as colcemid, colchicine, paclitaxel, vinblastine or vincristine, (ix) kinase inhibitors such as flavopiridol, staurosporin, STI571 (CPG 57148B) or UCN-01 (7-hydroxystaurosporine), (x) miscellaneous investigational agents such as thioplatin, PS-341, phenylbutyrate, ET-18-OCH.sub.3, or farnesyl transferase inhibitors (L-739749, L-744832), polyphenols such as quercetin, resveratrol, piceatannol, epigallocatechine gallate, theaflavins, flavanols, procyanidins, betulinic acid and derivatives thereof, (xi) hormones such as glucocorticoids or fenretinide, (xii) hormone antagonists, such as tamoxifen, finasteride or LHRH antagonists. Other adjuvants include agents able to sensitize for or induce apoptosis by binding to death receptors (“death receptor agonists”). Such agonists of death receptors include death receptor ligands such as tumor necrosis factor .alpha. ITNF-.alpha.), tumor necrosis factor .beta. (TNF-.beta., lymphotoxin-.alpha.), LT-.beta. (lymphotoxin-.beta.), TRAIL (Apo2L, DR4 ligand), CD95 (Fas, APO-1) ligand, TRAMP (DR3, Apo-3) ligand, DR6 ligand as well as fragments and derivatives of any of said ligands. Preferably, the death receptor ligand is TNF-.alpha.

The nucleic acid of the present invention can be administered alone or in combination with radiation and/or one or more active compounds. The latter can be administered before, after or simultaneously with the administration of the nucleic acid. The dose of either the nucleic acid or the active compound as well as the duration and the temperature of incubation can be variable and depends on the target that is to be treated. A further object of the present invention are pharmaceutical preparations which comprise an effective dose of at least one nucleic acid and/or at least one active compound and a pharmaceutically acceptable carrier, i.e. one or more pharmaceutically acceptable carrier substances and/or additives. The pharmaceutical according to the invention can be administered orally, for example in the form of pills, tablets, lacquered tablets, sugar-coated tablets, granules, hard and soft gelatin capsules, aqueous, alcoholic or oily solutions, syrups, emulsions or suspensions, or rectally, for example in the form of suppositories. Administration can also be carried out parenterally, for example subcutaneously, intramuscularly or intravenously in the form of solutions for injection or infusion. Other suitable administration forms are, for example, percutaneous or topical administration, for example in the form of ointments, tinctures, sprays or transdermal therapeutic systems, or the inhalative administration in the form of nasal sprays or aerosol mixtures, or, for example, microcapsules, implants or rods. The preferred administration form depends, for example, on the disease to be treated and on its severity. The preparation of the pharmaceutical compositions can be carried out in a manner known per se. To this end, the nucleic acid and/or the active compound, together with one or more solid or liquid pharmaceutical carrier substances and/or additives (or auxiliary substances) and, if desired, in combination with other pharmaceutically active compounds having therapeutic or prophylactic action, are brought into a suitable administration form or dosage form which can then be used as a pharmaceutical in human or veterinary medicine. For the production of pills, tablets, sugar-coated tablets and hard gelatin capsules it is possible to use, for example, lactose, starch, for example maize starch, or starch derivatives, talc, stearic acid or its salts, etc. Carriers for soft gelatin capsules and suppositories are, for example, fats, waxes, semisolid and liquid polyols, natural or hardened oils, etc. Suitable carriers for the preparation of solutions, for example of solutions for injection, or of emulsions or syrups are, for example, water, physiological sodium chloride solution, alcohols such as ethanol, glycerol, polyols, sucrose, invert sugar, glucose, mannitol, vegetable oils, etc. It is also possible to lyophilize the nucleic acid and/or the active compound and to use the resulting lyophilizates, for example, for preparing preparations for injection or infusion. Suitable carriers for microcapsules, implants or rods are, for example, copolymers of glycolic acid and lactic acid. The pharmaceutical preparations can also contain additives, for example fillers, disintegrants, binders, lubricants, wetting agents, stabilizers, emulsifiers, dispersants, preservatives, sweeteners, colorants, flavorings, aromatizers, thickeners, diluents, buffer substances, solvents, solubilizers, agents for achieving a depot effect, salts for altering the osmotic pressure, coating agents or antioxidants. The dosage of the nucleic acid, in combination with one or more active compounds to be administered, depends on the individual case and is, as is customary, to be adapted to the individual circumstances to achieve an optimum effect. Thus, it depends on the nature and the severity of the disorder to be treated, and also on the sex, age, weight and individual responsiveness of the human or animal to be treated, on the efficacy and duration of action of the compounds used, on whether the therapy is acute or chronic or prophylactic, or on whether other active compounds are administered in addition to the nucleic acid.

In some embodiments the immune cell surface protein aptamer is a PD-1 binding aptamer. PD-1 binding aptamers are described in, Prodeus et al Molecular Therapy Nucleic Acids (2015) 4 e237, Ti-Hsuan Ku Sensors 2015, 15, 16281-16313, and WO2016/019270, each specifically incorporated herein by reference. In some embodiments the immune cell surface protein aptamer is a CTLA4 binding aptamer. CTLA4 binding aptamers are described in Herrmann et al., J Clin Invest. 2014; 124(7):2977-2987, Gilboa et al., Clin Cancer Res; 19(5); 1054-62, and Santulli-Marotto et al., Cancer Res. 2003 Nov. 1; 63(21):7483-9, each specifically incorporated herein by reference. Aptamers are normally mono-specific, i.e. having high affinity and specificity for a single target molecule. The nucleic acid sequence of a mono-specific aptamer, or mono-specific part of a bi-specific aptamer, according to the present invention may optionally have a minimum length of one of 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 nucleotides

The nucleic acid sequence of a mono-specific aptamer, or mono-specific part of a bi-specific aptamer, according to the present invention may optionally have a maximum length of one of 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 nucleotides.

The nucleic acid sequence of a mono-specific aptamer, or mono-specific part of a bi-specific aptamer, according to the present invention may optionally have a length of one of 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 nucleotides.

The nucleic acid sequence of a mono-specific aptamer or mono-specific part of a bi-specific aptamer (including when present in a bi-specific aptamer complex), according to the present invention may have a degree of primary sequence identity with one of SEQ ID NOs 1 to 24 or 28 to 32, that is at least one of 80%, 81%, 82%, 83%, 84%, 85%, 86%, 87%, 88%, 89%, 90%, 91%, 92%, 93%, 94%, 95%, 96%, 97%, 98%, 99%, or 100%.

A “bi-specific aptamer” is an aptamer-based compound or composition that has high affinity and specificity for two, or at least two, different target molecules. A bi-specific aptamer may be comprised of the nucleic acid sequence of two mono-specific aptamers. A bi-specific aptamer may be a complex or conjugate of two mono-specific aptamers. The nucleic acid sequences of the two mono-specific aptamers may be brought together to form a complex, which may be a covalent or non-covalent complex. In some embodiments the bi-specific aptamer may comprise the nucleic acid sequence of a tumor cell antigen aptamer in complex with the nucleic acid sequence of an immune cell surface protein aptamer. As such, a bi-specific aptamer may be a complex of a tumor cell antigen binding moiety and an immune cell surface protein binding moiety. A covalent complex may be provided by forming a covalent bond between members of the complex. In some embodiments a bi-specific aptamer may be formed by synthesizing a single oligonucleotide molecule that comprises the nucleic acid sequence of a first mono-specific aptamer followed by the nucleic acid sequence of a second mono-specific aptamer, optionally with a linker between the two sequences. The linker may comprise one or more of an oligonucleotide sequence, hydrocarbon spacer elements such as optionally substituted C.sub.1-30 alkyl or optionally substituted C.sub.2-30 alkenyl; or polyethylene glycol molecule(s). In some embodiments the linker may be a polycarbon linker, consistent with formation of a “sticky bridge”. The polycarbon linker may be an optionally substituted C.sub.10-30 alkyl, optionally substituted C.sub.10-15 alkyl, optionally substituted C.sub.15-20 alkyl, optionally substituted C.sub.20-25 alkyl, optionally substituted C.sub.25-30 alkyl, optionally substituted C.sub.10-30 alkenyl, optionally substituted C.sub.10-15 alkenyl, optionally substituted C.sub.15-20 alkenyl, optionally substituted C.sub.20-25 alkenyl, optionally substituted C.sub.25-30 alkenyl.

A non-covalent complex may be provided by forming one or more non-covalent bonds between members of the complex. Non-covalent complexes may be maintained by hydrogen bonding, van der Waal forces and optionally ionic interaction. In some embodiments a bi-specific aptamer may be formed by attaching one of a pair of linker moieties to the nucleic acid sequence of each of two mono-specific aptamers, where the linker moieties have affinity or complementarity for each other, and allowing the linker moieties to bind and form a non-covalent complex. Examples of suitable linker moieties include a pair of single stranded oligonucleotides having complementary sequences that permit hybridization or tag and capture element pairs such as biotin and avidin/streptavidin. As used herein, the term “conjugate,” “bioconjugate” or “bioconjugate reactive group” or “bioconjugate linker” refers to the association between atoms or molecules. The association can be direct or indirect. For example, a conjugate between a first moiety (e.g. —NH.sub.2, —COOH, —N-hydroxysuccinimide, or -maleimide) and a second moiety (e.g., sulfhydryl, sulfur-containing amino acid) provided herein can be direct, e.g., by covalent bond or linker, or indirect, e.g., by non-covalent bond (e.g. electrostatic interactions (e.g. ionic bond, hydrogen bond, halogen bond), van der Waals interactions (e.g. dipole-dipole, dipole-induced dipole, London dispersion), ring stacking (pi effects), hydrophobic interactions and the like). In embodiments, conjugates are formed using conjugate chemistry including, but are not limited to nucleophilic substitutions (e.g., reactions of amines and alcohols with acyl halides, active esters), electrophilic substitutions (e.g., enamine reactions) and additions to carbon-carbon and carbon-heteroatom multiple bonds (e.g., Michael reaction, Diels-Alder addition). These and other useful reactions are discussed in, for example, March, Advanced Organic Chemistry, 3rd Ed., John Wiley & Sons, New York, 1985; Hermanson, Bioconjugate Techniques, Academic Press, San Diego, 1996; and Feeney et al., Modification of Proteins; Advances in Chemistry Series, Vol. 198, American Chemical Society, Washington, D.C., 1982. In embodiments, the first moiety (e.g., a tumor cell antigen binding moiety) is non-covalently attached to the second moiety on the immune cell surface protein binding moiety through a non-covalent chemical linker or covalent chemical linker formed by a reaction between a component of the first moiety and a component of the second moiety. In embodiments, the first moiety (e.g., a tumor cell antigen binding moiety) includes one or more reactive moieties, e.g., a covalent reactive moiety, as described herein (e.g., alkyne, azide, amine, ester, N-hydroxy-succinimide, maleimide or thiol reactive moiety). In embodiments, the first moiety (e.g., a tumor cell antigen binding moiety) includes a linker (e.g., first linker) with one or more reactive moieties, e.g., a covalent reactive moiety, as described herein (e.g., alkyne, azide, amine, ester, N-hydroxy-succinimide, maleimide or thiol reactive moiety). In embodiments, the second moiety (e.g., an immune cell surface protein binding moiety) includes one or more reactive moieties, e.g., a covalent reactive moiety, as described herein (e.g., alkyne, azide, amine, ester, N-hydroxy-succinimide, maleimide or thiol reactive moiety). In embodiments, the second moiety (e.g., an immune cell surface protein binding moiety) includes a linker with one or more reactive moieties, e.g., a covalent reactive moiety, as described herein (e.g., alkyne, azide, amine, ester, N-hydroxy-succinimide, maleimide or thiol reactive moiety).

Useful reactive moieties or functional groups used for conjugate chemistries herein include, for example: (a) carboxyl groups and various derivatives thereof including, but not limited to, Nhydroxysuccinimide esters, N-hydroxybenztriazole esters, acid halides, acyl imidazoles, thioesters, p-nitrophenyl esters, alkyl, alkenyl, alkynyl and aromatic esters; (b) hydroxyl groups which can be converted to esters, ethers, aldehydes, etc. (c) halo alkyl groups wherein the halide can be later displaced with a nucleophilic group such as, for example, an amine, a carboxylate anion, thiol anion, carbanion, or an alkoxide ion, thereby resulting in the covalent attachment of a new group at the site of the halogen atom; (d) dienophile groups which are capable of participating in Diels-Alder reactions such as, for example, maleimido groups; (e) aldehyde or ketone groups such that subsequent derivatization is possible via formation of carbonyl derivatives such as, for example, imines, hydrazones, semicarbazones or oximes, or via such mechanisms as Grignard addition or alkyllithium addition; (f) sulfonyl halide groups for subsequent reaction with amines, for example, to form sulfonamides; (g) thiol groups, which can be converted to disulfides, reacted with acyl halides, or bonded to metals such as gold; (h) amine or sulfhydryl groups, which can be, for example, acylated, alkylated or oxidized; (i) alkenes, which can undergo, for example, cycloadditions, acylation, Michael addition, etc; (j) epoxides, which can react with, for example, amines and hydroxyl compounds; (k) phosphoramidites and other standard functional groups useful in nucleic acid synthesis; (l) metal silicon oxide bonding; (m) metal bonding to reactive phosphorus groups (e.g. phosphines) to form, for example, phosphate diester bonds; and (n) sulfones, for example, vinyl sulfone.

The reactive functional groups can be chosen such that they do not participate in, or interfere with, the chemical stability of the proteins described herein. By way of example, the nucleic acids can include a vinyl sulfone or other reactive moiety. Optionally, the nucleic acids can include a reactive moiety having the formula S—S—R. R can be, for example, a protecting group. Optionally, R is hexanol. As used herein, the term hexanol includes compounds with the formula C6H130H and includes, 1-hexanol, 2-hexanol, 3-hexanol, 2-methyl-1-pentanol, 3-methyl-1-pentanol, 4-methyl-1-pentanol, 2-methyl-2-pentanol, 3-methyl-2-pentanol, 4-methyl-2-pentanol, 2-methyl-3-pentanol, 3-methyl-3-pentanol, 2,2-dimethyl-1-butanol, 2,3-dimethyl-1-butanol, 3,3-dimethyl-1-butanol, 2,3-dimethyl-2-butanol, 3,3-dimethyl-2-butanol, and 2-ethyl-1-butanol. Optionally, R is 1-hexanol.

The nucleic acid according to the present invention, respectively the medicaments containing the latter, optionally in combination with one or more active compounds can be used for the treatment of all cancer types which are resistant to apoptosis due to the expression of oncogenes and/or checkpoint inhibitors. Examples of such cancer types comprise neuroblastoma, intestine carcinoma such as rectum carcinoma, colon carcinoma, familial adenomatous polyposis carcinoma and hereditary non-polyposis colorectal cancer, esophageal carcinoma, labial carcinoma, larynx carcinoma, hypopharynx carcinoma, tong carcinoma, salivary gland carcinoma, gastric carcinoma, adenocarcinoma, medullary thyroid carcinoma, papillary thyroid carcinoma, follicular thyroid carcinoma, anaplastic thyroid carcinoma, renal carcinoma, kidney parenchyma carcinoma, ovarian carcinoma, cervix carcinoma, uterine corpus carcinoma, endometrium carcinoma, chorion carcinoma, pancreatic carcinoma, prostate carcinoma, testis carcinoma, breast carcinoma, urinary carcinoma, melanoma, brain tumors such as glioblastoma, astrocytoma, meningioma, medulloblastoma and peripheral neuroectodermal tumors, Hodgkin lymphoma, non-Hodgkin lymphoma, Burkitt lymphoma, acute lymphatic leukemia (ALL), chronic lymphatic leukemia (CLL), acute myeloid leukemia (AML), chronic myeloid leukemia (CML), adult T-cell leukemia lymphoma, hepatocellular carcinoma, gall bladder carcinoma, bronchial carcinoma, small cell lung carcinoma, non-small cell lung carcinoma, multiple myeloma, basalioma, teratoma, retinoblastoma, choroidal melanoma, seminoma, rhabdomyosarcoma, craniopharyngeoma, osteosarcoma, chondrosarcoma, myosarcoma, liposarcoma, fibrosarcoma, Ewing sarcoma and plasmacytoma.

Examples of cancer types where the use of the aptamers according to the present invention, respectively the medicaments containing the latter, is particularly advantageous include cervical carcinoma and melanoma.

1. An aptamer or aptamer-like composition capable of binding to a molecule associated with inhibition of immunological activity in a manner as to inhibit activity of said molecule associated with inhibition of immunological activity, while concurrently possessing a nucleic acid sequence capable of generating a molecule or series of molecules which induce RNA interference towards cancer promoting and/or immune suppressive molecules.

2. The aptamer of claim 1, wherein said molecule associated with inhibition of immunological activity is selected from the group consisting of: a) Adenosine A2B receptor, b) CD276, c) VTCN1, d) BTLA, e) CTLA-4, f) Indolamine 2,3 dioxygenase, g) Killer-cell Immunoglobulin-like Receptor, h) LAG-3, i) NOX2, j) PD-1, k) PD-L1, l) TIM3, m) VISTA, n) ILT-3, o) ILT-4, p) TGF-beta receptor, q) IL-10 receptor, r) IL-4 receptor, s) IL-13 receptor, t) IL-17 receptor, u) IL-20 receptor v) IL-24 receptor, w) IL-27 receptor, x) IL-35 receptor, y) Siglec-3, z) Siglec-5, aa) Siglec-7, ab) galectin-1, ac) galectin-3, ad) galectin-9, ae) FC gamma receptor b, af) GP49.

3. The aptamer of claim 1, wherein said nucleic acid sequence capable of generating a molecule or series of molecules are DNA molecules which are capable of transcribing short hairpin RNA.

4. The aptamer of claim 3, wherein said short hairpin RNA contains a stem loop.

5. The aptamer of claim 3, wherein said short hairpin RNA induces RNA interference.

6. The aptamer of claim 4, wherein said RNA interference is induced to inhibit expression of genes selected from the group consisting of: a) survivin, b) livin, c) CD161, and d) CD137.

7. The aptamer of claim 4, wherein said RNA interference is induced to inhibit expression of an oncogene.

8. The aptamer of claim 7, wherein said oncogene is selected from the group consisting of: a) abl, b) Af4/hrx, c) akt-2, d) alk, e) alk/npm, f) aml1, g) aml1/mtg8, h) bcl-2, 3, 6, i) bcr/abl, j) c-myc, k) dbl, l) dek/can, m) E2A/pbx1, n) egfr, o) enl/hrx, p) erg/TLS, q) erbB, r) erbB-2, s) ets-1, t) ews/fli-1, u) fms, v) fos, w) fps, x) gli, y) gsp, z) gsp, aa) HER2/new, ab) hox11, ac) hst, ad) IL-3, ae) int-2, af) jun, ag) kit, ah) KS3, ai) K-sam, aj) Lbc, ak) lck, al) Imo1, Imo-2, am) L-myc, an) lyl-1, ao) lyt-10, ap) lyt-10/C alpha 1, aq) mas, ar) mdm-2, as) mll, at) mos, au) mtg8/aml1, av) myb, aw) MYH11, ax) new, ay) N-myc, az) ost, ba) pax-5, bb) pbx1/E2a, bc) pim-1, bd) PRAD-1, be) raf, bf) RAR/PML, bg) Ras H, K, N, bh) rel/nrg, bi) ret, bj) rhom1, rhom2, bk) ros, bl) ski, bm) sis, bn) set/can, bo) src, bp) Tal1, tal2, bq) tan-1, br) Tiam1, bs) TSC2, and bt) trk.

9. The aptamer of claim 1, wherein CpG motifs are enriched in said aptamer so as to increase immunogenicity of said aptamer.

10. The aptamer of claim 9, wherein said CpG motifs activate TLR9.

11. The aptamer of claim 1, wherein said nucleic acid sequence capable of generating a molecule or series of molecules is DNA that encodes an RNA molecule in whole or in part expressing the sequence at least 10% homology to the sequence: CGGCAAGCAUUACGGUGUC (SEQ ID NO: 1) capable of inhibiting NR2F6.

12. The aptamer of claim 1, wherein said nucleic acid sequence capable of generating a molecule or series of molecules is DNA that encodes an RNA molecule in whole or in part expressing the sequence at least 10% homology to the sequence: GCAUCGACAACGUGUGCGA (SEQ ID NO: 2) capable of inhibiting NR2F6.

13. The aptamer of claim 1, wherein said nucleic acid sequence capable of generating a molecule or series of molecules is DNA that encodes an RNA molecule in whole or in part expressing the sequence at least 10% homology to the sequence: CUGGUUCCCCAGCUGUCAG (SEQ ID NO: 3) capable of inhibiting livin.

14. The aptamer of claim 1, wherein said nucleic acid sequence capable of generating a molecule or series of molecules is DNA that encodes an RNA molecule in whole or in part expressing the sequence at least 10% homology to the sequence: GGAAGAGACUUUGUCCACA (SEQ ID NO: 4) capable of inhibiting livin.

15. The aptamer of claim 1, wherein said nucleic acid sequence capable of generating a molecule or series of molecules is DNA that encodes an RNA molecule in whole or in part expressing the sequence at least 10% homology to the sequence: GGAAGAGACTTTGTCCACAT (SEQ ID NO: 5) capable of inhibiting survivin.

16. The aptamer of claim 1, wherein said molecules are used in conjunction with an NF-kappa B inhibitor.

17. The aptamer of claim 1, wherein said molecules are used in conjunction with an IKK inhibitor.

18. The aptamer of claim 1, wherein said molecules are used in conjunction with a MAP kinase inhibitor.

19. The aptamer of claim 1, wherein said molecules are used in conjunction with a BORIS inhibitor.

20. The aptamer of claim 1, wherein said molecules are used in conjunction with a toll like receptor inhibitor.
